(19) United States
(12) Plant Patent Application Publication
(10) Pub. No.: US 2004/0139522 P1
(43) Pub. Date: Jul. 15, 2004

(54) Osteospermum plant named 'Osjotis'

(76) Inventor: Johannes Simon Nicolaas Oud, Bovenkarspel (NL)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/342,638

(22) Filed: Jan. 15, 2003

(51) Int. Cl. A01H 5/00

(52) U.S. Cl. PLT/360

(57) ABSTRACT

A new Osteopermum plant particularly distinguished by its very compact round plant habit, early flowering with light purple colored ray floret and blue colored disc floret.

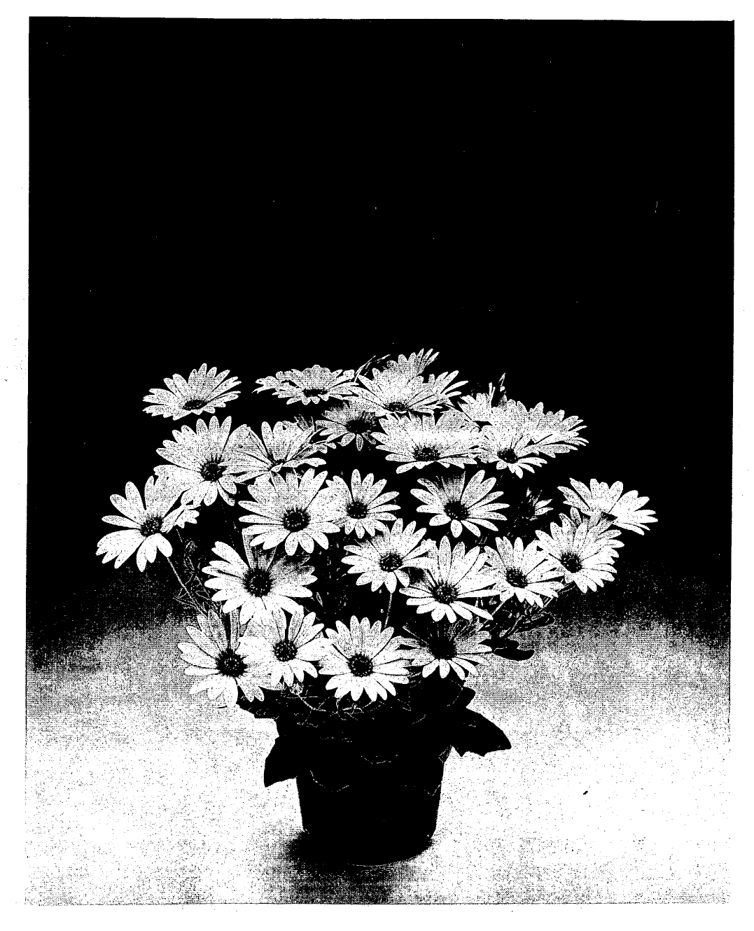

### VARIETAL DENOMINATION

‘Osjotis’

### BACKGROUND OF THE NEW PLANT

The present invention relates to a new and distinct cultivar of Osteopermum plant, botantially known as Osteopermum ecklonis. The new Osteopermum is propagated by cuttings resulting from a cross of a proprietary Osteospermum ecklonis seedling selection as the male, or pollen, parent identified as code number ‘C274’ with a proprietary Osteospermum ecklonis selection identified as code number ‘C298’ as the female, or seed, parent.

The new Osteopermum was discovered and selected as a single flowering plant within the progeny of the stated cross grown in Enkhuizen, Netherlands, in 1998. Neither ‘C274’ nor ‘C298’ has been patented. The new Osteopermum plant has been repeatably asexually reproduced by cuttings in Enkhuizen, Netherlands over a period of 3 years. It has been found to retain its distinctive characteristics through successive propagations, and this novelty appears to be firmly fixed.

### DESCRIPTION OF THE DRAWING

This new Osteopermum plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Osteopermum plant. The data which defines these characteristics were collected from asexual reproductions carried out in Enkhuizen, Netherlands. The plant history was taken on 15 weeks old plants. Color references are primarily to the RHS Colour Chart of The Royal Horticultural Society of London.

Comparison between the new cultivar ‘Osjotis’ and
a similar cultivar

| | ‘Osjotis’ | ‘Sea side’ |
|---|---|---|
| Plant height | 20 cm | 30 cm |
| Plant width | 15 cm | 20 cm |
| Earliness | | Two weeks |
| | | later flowering |

The plant:

Classification.—Botanical: Osteopermum ecklonis.

Parentage:

Female parent.—A seedling named ‘C298’ is one of our seedlings from our C-generation of plants bred in 1998.

Male parent.—A seedling named ‘C274’ is one of our seedlings from our C-generation of plants bred in 1998.

Plant description:

Growth habit.—Round, compact.

Plant height.—20 cm.

Speading area of plant.—15 cm.

Strength.—Moderate growth

Branching character.—Freely branching, 20 lateral branches without pinching.

Blooming period.—From May until October.

The stem:

Length.—10 cm.

Diameter.—4 mm.

Internode length.—8 mm.

Texture.—Glabrous.

Color.—RHS 137C.

The foliage:

Shape.—Single leaves, alternate and elliptic, apex: broadly acute base: attenuate, margin: irregular serration.

Number of leaves per lateral branch.—13.

Texture.—Glabrous, leathery.

Leaf.—Color: RHS 137B — Length: Young 30 mm, mature 70 mm — Width: Young 10 mm, mature 40 mm.

The flower:

Number of flowers per mature plant.—100.

Flower bud.—Form: Ovoid — Length: 20 mm — Width: 12 mm — Color: RHS N79D.

Flower.—Diameter: 63 mm — Disc diameter: 9 mm.

Disc florets.—Shape: Tubular, elongated — Apex: Five pointed — Length: 7 mm — Width: 1 mm Number of disc florets per inflorescence: 60 — Color: RHS 99B.

Ray florets.—Length: 27 mm — Width: 7 mm — Color upper surface: RHS 77C. lower surface: RHS N79D — Number of ray florets: 20 — Shape — Apex: Rounded — Base: Acute — Margin: Entire — Texture: Satiny.

Peduncles.—Length: 8 cm — Texture: Sturdy, tiny hairs — Color: RHS 137C.

Sepals.—Texture: Rough, tiny hairs — Number of sepals: 20 — Color: RHS 137C — Length: 10 mm.

Reproductive organs:

Androecium.—Appearance: Present on disc florets only — Shape: Oblong, five stamen fused to one — Length: 2 mm — Width: 1 mm — Pollen color: RHS 25A — Total no. of anthers: 60.

Gynoecium.—Appearance: Present on both ray and disc florets — Pistil number: One pistil per ray or disc floret — Pistil length: 4 mm — Stigma color: RHS 77A — Stigma shape: Bipartite.

Seed development: Seed development has been observed on plants of the new Osteopermum.

Disease resistance: Under commercial conditions, plants of the new Osteopermum are resistant to fungal pathogens common to Osteopermum.

What is claimed is:

1. A new and distinct variety of Osteopermum plant, substantially as illustrated and described herein.
